(19) United States
(12) Patent Application Publication
(10) Pub. No.: US 2023/0293199 A1
(43) Pub. Date: Sep. 21, 2023

(54) DETERMINING A MOVEMENT OF AN OBJECT, AND THERAPY APPARATUS

(71) Applicant: Siemens Healthcare GmbH, Erlangen (DE)

(72) Inventors: Tobias Lenich, Nürnberg (DE); Alois Regensburger, Poxdorf (DE)

(21) Appl. No.: 18/121,623

(22) Filed: Mar. 15, 2023

(30) Foreign Application Priority Data
Mar. 15, 2022 (DE) 10 2022 202 553.9

(51) Int. Cl. A61B 17/225; A61B 8/08; A61B 8/00; A61B 18/12

(52) U.S. Cl. CPC A61B 17/2255; A61B 8/085; A61B 8/481; A61B 8/54; A61B 18/12

(57) ABSTRACT

The movement of an object, in particular of an organ during histotripsy treatment is determined specifically for repositioning purposes. This is done by producing bubbles in or at the object. The bubbles are detected, and their movement is recorded. The movement of the object is estimated from their movement. If applicable, a focal point of a therapy apparatus may be repositioned in accordance with the movement of the object.

### CROSS REFERENCE TO RELATED APPLICATIONS

This application claims the benefit of DE 102022202553.9 filed on Mar. 15, 2022, which is hereby incorporated by reference in its entirety.

### FIELD

Embodiments relate to a method for determining a movement of an object or an organ or tissue of a patient.

### BACKGROUND

Certain therapy methods are based on using waves or radiation to treat an organ. As a rule, in these methods, it is important to apply the waves or radiation with pinpoint accuracy. For instance, for histotripsy, it is essential to ablate tissue at a precisely specified focal point. Similarly, for lithotripsy, it is vital that the high energy shock waves hit a kidney stone or a gallstone in the center. The same applies to ablation methods, in which the sound or radiation must be directed precisely onto a specific tissue segment.

The human and animal organism, however, is characterized by numerous movements. For example, respiratory and cardiac movements cause also other organs inside the same body to move as well. If organs that are moving or caused to move are now meant to be treated by waves or radiation, it may be necessary to correct or compensate for the various movements. In complex therapy methods in which, for example, a focal point of the radiation or of the shock waves is moved along a defined path over a prolonged period, it may be desirable to correct the movement of the organ concerned online. For instance, online movement correction is required for precise and targeted emission of histotripsy ultrasound. It is very difficult in this process, however, to record internal movements of organs and tissue accurately.

There are a number of motion correction methods, that are based on various imaging modalities (external or internal). All these modalities, however, would require additional registration with the histotripsy ultrasonic-probe or transducer. The imaging transducer of the histotripsy instrument may be used to record tissue movements and/or organ movements by ultrasound. This may usually be achieved only by additional contrast agents and/or marker structures, however.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments determine the movement of an object in a simplified manner. A corresponding method and a suitable therapy apparatus is provided.

According to an embodiment, a method for determining a movement of an object is provided, in which method bubbles (for example gas, vapor or vacuum bubbles) are produced in or at the object. The intention is hence to ascertain or determine the movement of the object. A measure for the movement may be, for example, the position of a point as a function of time. The object may also be characterized by a plurality of points, however, that move individually as a function of time, i.e., the plurality of points' respective positions change individually. Movements may also be recorded in the form of speeds and directions, however. The exact measure of the movement is not relevant here.

The object itself may be of any type. For example, the object is an organ such as the liver. It is also possible, however, that the object is other organs or portions of organs. For example, the object may be a tissue segment, and may possibly be a tumor. It is also possible, however, that the object is a foreign body in an organism, for instance a kidney stone, or even a moving object in a technical system.

Waves may be transmitted into or onto the object. For example, electromagnetic radiation is applied into or onto the object. For example, the electromagnetic radiation may be visible light or even infrared radiation that is directed at the object. Alternatively, the waves may also be acoustic waves that are directed at the object. It is also possible for example here that ultrasound waves are used to act on the object. The waves reach the surface of the object or penetrate deeper into the object.

For example, bubbles that have no therapeutic effect and do not damage tissue are produced in or at the object by technical action. This means, for example, that the waves interact with the object. Specifically, the energy of the waves may be used to produce bubbles. For example, bubbles may appear as a result of vaporization of tissue fluid. Alternatively, however, they may also appear as a result of negative pressure, as is the case in histotripsy. In this case, vacuum cavitations are formed, for example. These bubbles may disappear again in a pressure phase. In the case of thermally produced bubbles, these may disappear again as a result of cooling.

In a further step of the method, detection of the bubbles in or at the object is carried out. This means that the bubbles must be recorded as such. Thus, the bubbles must be identified, that is possible by suitable image processing, for example. The bubbles must be detected in or at the object. In the object means that the bubbles must be located underneath the surface or within the surface of the object. At the object means that the bubbles are touching the surface of the object or are at a maximum distance of one centimeter from the object. It may be assumed for these small distances that the bubbles follow the movement of the object.

In a further step of the method for determining the movement of the object, a movement of the bubbles is recorded. The movement of the bubbles may be determined, for example, by observing their shape or position as a function of time. A movement of the bubbles per se is less relevant here, however. What is of primary interest, however, is the movement of the bubbles for example relative to the transmit device that is emitting the waves, or relative to the propagation geometry of the waves, for instance a focal point of the electromagnetic beams or of the shock waves. For example, the bubbles are bound in a tissue matrix, and do not change their location relative to the tissue or change it only negligibly. If now the tissue moves relative to the transmitter of the waves, the bubbles in the tissue also move as well accordingly. Conversely, a movement of the tissue may be inferred from the movement of the bubbles.

In the method, an estimated value for the movement of the object is accordingly obtained from the movement of the bubbles. If, for example, the bubbles are located in the object, it may be assumed that the bubbles and the object move synchronously with each other. If the bubbles are located directly on the object or at a small distance from the object, that may be the case for tissue ablations, for instance, then it may be assumed, for example, that the movement of the bubbles is largely similar to a movement of the object. The estimated value for the movement may take into account, for example, distances of the bubbles from the object, or fluid properties from the area surrounding the object. Whatever the case, the movement of the object is inferred from the movement of the bubbles. If applicable, a suitable movement measure is provided for the movement of the object. Easily detectable bubbles may thereby be used advantageously to record or determine the movement of an object.

According to an embodiment, producing the bubbles is carried out by introducing energy into or onto the object. For example, the energy may be introduced by ultrasound waves. The object is thus exposed to ultrasound waves, that for example may be pulsed. Shock waves, for example, may be generated in this manner. Ultrasound refers to sound of frequencies above the frequency range audible to humans. It includes frequencies from 20 kHz to 10 GHz. Suitably high-energy ultrasound waves may be used, for example, to break up kidney stones or to achieve ablations at tissues. In the present case, the ultrasound may also be used to produce the bubbles in the tissue or in the organ in order to record its movement. For example, in histotripsy, ultrasound is used to produce the relevant cavitations in the tissue.

In an embodiment, the energy for producing the bubbles may be introduced by electromagnetic waves. For example, these may be radio waves or microwaves. These waves are suitable for penetrating suitably deep into tissue. Microwaves are electromagnetic waves of frequency in the band 300 MHz to about 1 THz, that corresponds to a wavelength of about 1 m to 0.3 mm. At the lower end of the microwave frequency band, i.e., towards lower frequencies, is the adjoining radio-wave band; towards the upper end is the infrared region of the optical spectrum. The radio waves and microwaves are likewise suitable for the ablation of tissue and may also be used to produce bubbles by locally overheating the tissue.

In an embodiment, the production of the bubbles is performed by injecting bubbles, or on the basis of a chemical reaction. For example, a corresponding device for producing the bubbles includes a catheter and/or injector in order to deliver the bubbles into or onto the object. If applicable, an injected substance lowers a cavitation threshold so that cavitation bubbles appear more easily. In the case of producing the bubbles by chemical reaction, a suitable reagent may be injected.

In an embodiment of the method, it may be provided that a first portion of the energy or waves is focused at a focal point in or at the object, and a second portion of the energy or waves, which portion differs from the first portion, produces the bubbles at another site away from the focal point. This means that the waves (also representative for energy below) include a power density maximum in a defined region in or at the object, and the bubbles outside the region are produced at a power density of the waves that is less than the power density maximum. In the case of histotripsy, this would mean that the waves include a power density maximum at the focal point, so that it is possible to destroy tissue there by the cavitations that are produced. Outside the focal point or power density maximum, however, although cavitations may still be produced, they basically have a negligible effect because they cause barely any or no tissue destruction. Nonetheless, the bubbles are of value for determining the movement of the organ or of the tissue portion.

According to an embodiment of the method, the focal point of the emitted waves is repositioned or switched on/switched off according to the estimated value for the movement of the object. The focal point may be repositioned exactly according to the estimated movement of the object. This means that despite the movement, for example the same portion of the object remains at the focal point of the waves. It is thereby possible to act very precisely on this object portion located at the focal point. For example, a robot arm, that, for instance, moves the ultrasonic probe of a histotripsy apparatus with it according to the movement of the object (organ), may be used for the repositioning. Alternatively, or additionally, the focal point may be repositioned electronically, for instance by a matrix transducer. If applicable, however, the introduction of the energy or waves may also simply be switched off when the object is moving too much, and possibly switched back on when there is little movement of the object.

In an embodiment, the treatment of the tissue by the energy or waves may take place at a different time from the production of the bubbles for the movement determination by the waves. It may thus be provided, for instance, that the waves are applied at a first power density in a first phase for producing the bubbles for the movement determination, and at a second power density in a second phase. The first power density is less than the second power density. The first phase and the second phase may be of equal length, although they may also include a different time length from each other and for example may follow directly one after the other in time. The power density may be changed easily by varying the power of the waves that are sent out.

Alternatively, the power density may also be regulated by changing the spatial exposure region by focusing or defocusing the waves or the beams accordingly. In the defocused state, bubbles that have no therapeutic effect and do not damage tissue are primarily produced, whereas in the focused state, tissue segments or objects may be destroyed accordingly. In an embodiment, it may thus be provided that the waves are defocused at a defined location in the first phase and are focused at the defined location in the second phase. In the case of histotripsy, this could mean that initially the movement of the object is recorded on the basis of small bubbles, and then at the same location, cells are destroyed with focused ultrasound waves.

The aforementioned object is also achieved by a recording apparatus for determining a movement of an object, including a production device for producing bubbles in or at the object, a detection device for detecting the bubbles in or at the object, a recording device for recording a movement of the bubbles, and an analysis device for obtaining an estimated value for the movement of the object from the movement of the bubbles.

In the case of histotripsy, the production device may include an ultrasonic probe, that may be used to send out focusable ultrasound. Also, in the case of HIFU (high-intensity focused ultrasound), LOFU (low-energy focused ultrasound) and for lithotripsy, the production device may likewise include a suitable ultrasonic probe. In the case of thermal ablation, the production device may also include a microwave transmitter. For photoablation, the production device may include an appropriate light emitter. It is also possible, however, that the production device has an injector or catheter for administering bubbles or reagents for producing bubbles.

The detection device for detecting the produced bubbles may be the same for all the emission techniques. For example, the bubbles may be observed always using ultrasound. Gas bubbles appear with very high contrast in the ultrasound image. In principle, however, the detection device may also be based on other physical principles such as X-ray absorption or magnetic resonance.

The analysis device for recording the movement of the bubbles and for obtaining the estimated value for the movement of the object may be based on a processing unit. For example, a processor that performs the corresponding data processing may be integrated in the analysis device.

A therapy apparatus may be equipped with the aforementioned recording apparatus. A component of the therapy apparatus is controlled according to the estimated value for the movement of the object. The component may be a repositioning device for repositioning a focal point of the production or emission device according to the estimated value for the movement of the object. The component may also be switching device, however.

The repositioning device may be controlled by a processor to reposition the focal point of the emission device. In an embodiment, the repositioning device is integrated in a control device of the therapy apparatus. For the control, the repositioning device receives the estimated value obtained by the analysis device and supplies a corresponding control signal to the emission device.

The possible variations and advantages mentioned above in connection with the method apply mutatis mutandis also to the therapy device. The aforementioned method steps may be regarded as functional features of the therapy apparatus.

In an embodiment, the therapy device is in the form of a histotripsy apparatus, thermal ablation apparatus or lithotripsy apparatus. The individual apparatuses are then each furnished with the aforementioned features.

In an embodiment, the therapy apparatus includes an imaging device (for example an imaging probe) for creating images of the object in the region of the focal point. The detection device for detecting the bubbles for determining the movement of the object is integrated in the imaging device. In this case, the therapy apparatus therefore has an imaging device that may be used to obtain from the object, images in the region of the focal point of the emission device. The therapy may hence be assisted by suitable imaging. It is precisely this imaging device that is now also used to observe or to detect the bubbles needed for determining the movement of the object. The imaging device includes algorithms that are specifically suited to detecting the bubbles, for example.

The aforementioned object is also achieved by a computer program, that may be loaded directly into a memory of a control device of the therapy apparatus and has program code configured to perform the steps of the aforementioned method when the program code is executed in the control device of the therapy apparatus. Likewise, there may be an electronically readable data storage medium including electronically readable control information stored thereon, which information includes at least one computer program (product) described and is configured such that it performs the described method when the data storage medium is used in a control device of a therapy apparatus.

### BRIEF DESCRIPTION OF THE FIGURES

FIG. 1 depicts a schematic view of a therapy apparatus for histotripsy according to an embodiment.

FIG. 2 depicts an acquisition of histotripsy bubbles according to an embodiment.

FIG. 3 depicts a schematic flow diagram of an embodiment of a method.

### DETAILED DESCRIPTION

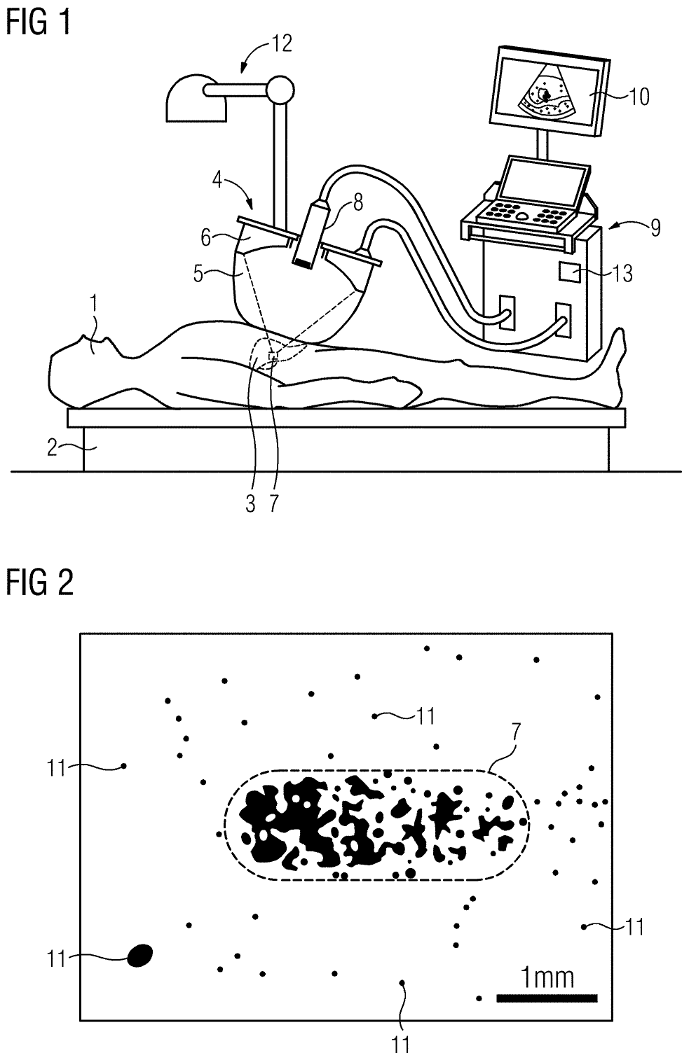

FIG. 1 depicts a patient 1 on a couch 2. The liver 3 of the patient 1 is being treated by histotripsy. For this purpose, a histotripsy transducer 4 is placed on the abdomen or ribcage of the patient 1. The histotripsy transducer 4 is approximately in the shape of a pot. In the region of the base and wall of the pot is a coupling segment 5 of the histotripsy transducer 4. On the side of the histotripsy transducer 4 that faces away from the patient 1 is arranged the actual ultrasound transducer 6 effectively as a pot lid. The ultrasound transducer 6 is designed to focus the emitted ultrasound waves in a focal region 7. In the present example, the focal region 7 lies at that site of the liver 3 that is to be treated.

The histotripsy transducer 4 may include an imaging probe 8, that may be used to obtain images from the focal region 7 and, if applicable, the area surrounding it. The imaging probe is a sonography instrument, for instance. It may be arranged in the center of the ultrasound transducer 6, and likewise acoustically coupled to the patient 1 via the coupling segment 5.

In the present example, the ultrasound transducer 6 constitutes at least a part of the emission device, that transmits waves (in this case ultrasound waves) into the liver 3 of the patient 1. The liver 3 therefore constitutes the object, the movement of which is meant to be determined. The movement of the liver 3 results, for instance, from the breathing and heartbeat of the patient 1. It may be the case, however, that movements of the liver originate from motor movements of the patient 1. If the patient specifically does not remain still in one spot during the treatment, but changes their position, for instance on the couch 2, then the position of their liver 3 with respect to the histotripsy transducer 4 also changes.

In the example of FIG. 1, both the actual ultrasound transducer 6 and the imaging probe 8 are controlled by a shared control device 9. The control device 9 may include a screen as an output unit 10. In addition, the control device has a detection device (not explicitly shown) and an analysis device (likewise not explicitly shown). The signals from the imaging probe 8 may be analyzed by the detection device in order to detect bubbles in or at the object (liver 3 in this case). The bubbles identified by the detection device are analyzed by the analysis device in terms of their movement. For example, movements of the bubbles that take place relative to the histotripsy transducer 4 are recorded. Purely a change in size of the bubbles is usually ignored here. As soon as the analysis device records a movement of the bubbles, this movement may be used as an estimated value for the movement of the object intended for therapy (liver 3 in this case). The movement of the object is thus registered indirectly by the bubbles. Additional equipment or an additional probe for recording the movement of the object is not necessary with the design. Instead, for example, the signals from a standard histotripsy apparatus that includes imaging may be used also to establish movements of the object intended for therapy. This design of the histotripsy apparatus including histotripsy transducer 4 and control device 9 may be applied to other therapy apparatuses as well, for instance those for lithotripsy, thermal ablation and the like.

FIG. 2 depicts an image of a focal region 7, that was obtained, for example, by the imaging probe 8 or a sonography instrument. Inside the focal region 7 may be seen numerous bubbles, that in the present case were produced by the histotripsy transducer 4. The focal region 7 is clearly demarcated from the surrounding area. Bubbles 11 also appear outside the focal region 7. The bubbles 11 outside the focal region 7 are generally smaller than inside the focal region 7. The reason for this is that the power density of the ultrasound waves is lower outside the focal region 7. Nonetheless, even the smaller bubbles 11 outside the focal region 7 may be detected and tracked. For example, for example, their movement relative to the focal region 7 may be recorded. Whereas large bubbles tend to appear and also disappear again only inside the focal region 7, small bubbles are produced outside the focal region 7 for a lower power density. With a movement of the organ or object, the small bubbles 11 also move relative to the focal region 7. Hence the small bubbles also move relative to the waves or to their direction of propagation, and also relative to the emission device (the histotripsy transducer in this case).

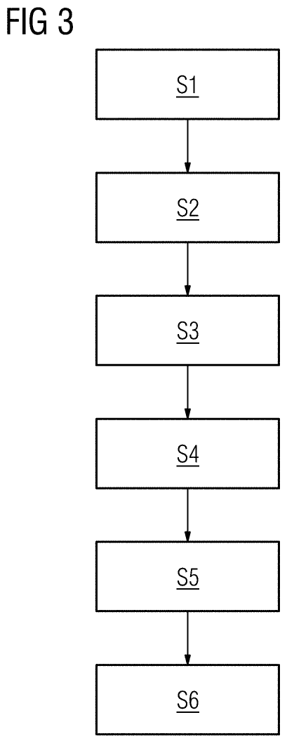

FIG. 3 is a schematic flow diagram of an embodiment of the method. In a first step S1, waves are transmitted into or onto an object. The waves may be ultrasound waves or may be electromagnetic waves such as radio waves or microwaves.

In a subsequent step S2, bubbles are produced in or at the object (by the waves). For example, the waves may be focused, so that there is a higher power density inside a focal region compared with a region outside the focal region. Nonetheless, as soon as a cavitation threshold, for instance in histotripsy, is exceeded, cavitation bubbles are expected to form in the region outside the focal region as well.

In a step S3, bubbles are detected in or at the object. These may be the bubbles outside the focal region.

In a step S4, a movement of the bubbles is recorded. For example, this movement may be recorded relative to the focal point or to the histotripsy transducer or to the particular therapy apparatus. Whereas the bubbles inside the focal region do not move detectably, the bubbles outside the focal region generally move with the object.

In a step S5, an estimated value for the movement of the object is obtained from the movement of the bubbles. Since the bubbles outside the focal region practically do not move relative to the object, the movement of the bubbles may be used as the estimated value for the movement of the object.

In an optional step S6, the focal point of the waves is repositioned according to the estimated value for the movement of the object. This repositioning may be performed using a robot arm (not shown in FIG. 1), that moves the histotripsy transducer 4 relative to the patient 1 or to their liver 3. This movement may be controlled by the control device 8. In this manner, the histotripsy transducer may be repositioned in accordance with the movement of the object (the liver 3 in this case). The repositioning in accordance with the movement may be performed irrespective of the cause of the movement (breathing, heartbeat, motor movement, etc.).

For a specific example, embodiments propose producing microbubbles outside a focal region or target area by unfocused histotripsy ultrasound that lies slightly above the cavitation threshold. For treating a tissue, for instance a tumor, by collapsing bubble cavitations, focused histotripsy ultrasound may be directed onto the relevant target area. Monitoring the bubble cloud produced in the focused histotripsy would not be sufficient to distinguish a movement of the tissue/organ relative to the histotripsy ultrasonic probe or histotripsy transducer because the bubble cloud and the ultrasound focal point remain stationary in relation to the ultrasonic probe. Microbubbles may be produced by using non-focused ultrasound outside the target area. The position of the microbubbles does not depend on the position/orientation of the ultrasonic probe because they are produced by unfocused ultrasound. When the tissue/organ moves inside the patient, the microbubbles move with the tissue/organ relative to the ultrasonic probe. Online motion-correction may be carried out by monitoring the position of the microbubbles relative to the ultrasonic probe, for instance by the imaging ultrasound probe. Advantageously, the focal point of the histotripsy ultrasound may be adjusted according to the ascertained microbubble position.

Embodiments thus advantageously provide for online motion-correction during a histotripsy treatment, in which the motion data, i.e., the detected microbubble position, is inherently registered with the histotripsy ultrasound probe.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining a movement of an object, the method comprising:
producing bubbles in or at the object;
detecting the bubbles in or at the object;
recording a movement of the bubbles; and
obtaining an estimated value for the movement of the object from the movement of the bubbles.

2. The method of claim 1, wherein producing the bubbles is carried out by introducing energy into or onto the object.

3. The method of claim 2, wherein introducing energy includes beaming in electromagnetic waves.

4. The method of claim 3, wherein the electromagnetic waves comprise at least one of radio waves, microwaves, or ultrasound waves.

5. The method of claim 1, wherein producing the bubbles is carried out by injecting the bubbles or by using a chemical reaction.

6. The method of claim 2, wherein a first portion of the energy is focused at a focal point in or at the object, and a second portion of the energy that differs from the first portion, produces the bubbles at another site away from the focal point without therapeutic effect.

7. The method of claim 6, wherein the focal point is repositioned or switched on/switched off according to the estimated value for the movement of the object.

8. The method of claim 2, wherein the energy is applied at a first power density in a first phase for producing the bubbles, and at a second power density in a second phase, wherein the first power density is less than the second power density.

9. The method of claim 8, wherein the energy is defocused at a defined location in the first phase and is focused at the defined location in the second phase.

10. The method of claim 1, further comprising:
controlling an ultrasound apparatus or an exposure apparatus according to the estimated value for the movement of the object.

11. A recording apparatus for determining a movement of an object. the recording apparatus comprising:
a production device for producing bubbles in or at the object;
a detection device for detecting the bubbles in or at the object;
a recording device for recording a movement of the bubbles; and
an analysis device for obtaining an estimated value for the movement of the object from the movement of the bubbles.

12. The recording apparatus of claim 11, wherein a component of a therapy apparatus is controlled according to the estimated value for the movement of the object.

13. The recording apparatus of claim 12, wherein the therapy apparatus comprises a histotripsy apparatus, a thermal ablation apparatus, or a lithotripsy apparatus.

14. The recording apparatus of claim 11, further comprising:
an imaging device configured for creating images of the object, wherein the detection device for detecting the bubbles for determining the movement of the object is integrated in the imaging device.

15. A non-transitory computer readable storage medium comprising a set of computer-readable instructions stored thereon for determining a movement of an object, the computer-readable instructions which, when executed by at least one processor cause the at least one processor to:
cause a production device to produce bubbles in or at the object;
detect the bubbles in or at the object;
record a movement of the bubbles; and
obtain an estimated value for the movement of the object from the movement of the bubbles.
